(19) United States
(12) Patent Application Publication
(10) Pub. No.: US 2023/0372629 A1
(43) Pub. Date: Nov. 23, 2023

(54) End-of-Dose Indicator for Medical Injection Device

(71) Applicant: Becton Dickinson France, Le Pont-de-Claix (FR)

(72) Inventors: Fabien Dubuc, Grenoble (FR); Julien Gagliano, Meylan (FR); Louis Petit, Venon (FR); Pierre Ducarouge, Milano (IT)

(21) Appl. No.: 18/031,395

(22) PCT Filed: Oct. 12, 2021
(86) PCT No.: PCT/EP2021/078224

§ 371 Date: Apr. 12, 2023

(30) Foreign Application Priority Data
Oct. 14, 2020 (EP) 20306206.2

(51) Int. Cl. A61M 5/315

(52) U.S. Cl. CPC A61M 5/3157; A61M 2205/583

(57) ABSTRACT

Provided herein is an end-of-dose indicator for a medical injection device including a housing having a proximal end, a distal end, and a sidewall therebetween defining an interior, the housing configured to be secured to a proximal end of a medical injection device. The indicator also includes a switch arranged within the housing interior and having an open position and a closed position, a circuit, and one or more indicators in electrical communication with the circuit. When in the closed position, the switch triggers the circuit to activate the indicator.

### CROSS-REFERENCE TO RELATED APPLICATION

The present application claims priority to European Application No. EP 20306206.2, entitled “End-Of-Dose Indicator for Medical Injection Device”, filed Oct. 14, 2020, the entire disclosure of which is hereby incorporated by reference in its entirety.

### BACKGROUND OF THE INVENTION

### Field of the Invention

The present disclosure relates generally to indicators for medical injection devices, and, in particular embodiments or aspects, to visual indicators providing an indication that a dose of a medicament has been completely delivered and the medical injection device can be removed from the site of injection.

### Description of Related Art

Medical injection devices often utilize indicators to inform users thereof that an injection has been completed. These indicators can take the form of audible, tactile, or visual indicators. However, in many cases the indicators are not sufficiently temporally delayed from the end of the dose, such that a user will remove the medical injection device from the site of injection before the entire dose of medicament has been delivered. Accordingly, there is a need in the art for a more robust indicator with a more reliable delay mechanism, such that full doses of a desired medicament is more reliably delivered.

### SUMMARY OF THE INVENTION

Provided herein is an end-of-dose indicator device for a medical injection device, having a housing having a proximal end, a distal end, and a sidewall therebetween defining an interior, the housing configured to be secured to a proximal end of a medical injection device, a switch arranged within the housing interior and having an open position and a closed position, a circuit, and one or more indicators in electrical communication with the circuit, wherein the switch, when in the closed position, triggers the circuit to activate the indicator.

Also provided herein is a medical injection system having a medical injection device including an injection device housing having a proximal end, a distal end, and a sidewall therebetween defining an injection device interior, a syringe received within the injection device interior, the syringe comprising a proximal end having a piston, a distal end having a needle, and a sidewall therebetween defining a syringe interior configured to receive a medicament, a plunger rod received within the injection device interior and configured to displace the piston distally to cause the medicament to be expelled through the needle, a drive assembly configured to displace the plunger rod distally, and a plunger rod follower having a distal end operatively coupled to the plunger rod and a proximal end. The system also includes an end-of-dose indicator device having a housing having a proximal end, a distal end, and a sidewall therebetween defining an interior, the housing configured to be secured to a proximal end of a medical injection device, a switch arranged within the housing interior and having an open position and a closed position, a circuit, and one or more indicators in electrical communication with the circuit, wherein the plunger rod follower is configured such when as the plunger rod is displaced distally the proximal end of the plunger rod follower is displaced proximally and contacts the switch, moving the switch from the open position to the closed position and triggering the circuit to activate the indicator.

Further embodiments or aspects are provided in the following numbered clauses:
Clause 1. An end-of-dose indicator device for a medical injection device, comprising: a housing comprising a proximal end, a distal end, and a sidewall therebetween defining an interior, the housing configured to be secured to a proximal end of a medical injection device; a switch arranged within the housing interior and having an open position and a closed position; a circuit; and one or more indicators in electrical communication with the circuit, wherein the switch, when in the closed position, triggers the circuit to activate the indicator.
Clause 2. The end-of-dose indicator device according to clause 1, wherein the housing comprises a cylinder with a flared proximal end.
Clause 3. The end-of-dose indicator device according to clause 2, wherein the flared proximal end comprises a proximal-facing outer surface configured to receive a user's thumb.
Clause 4. The end-of-dose indicator device according to any of clauses 1-3, wherein the one or more indicators are one or more visual indicators.
Clause 5. The end-of-dose indicator device according to clause 4, wherein the one or more visual indicators are one or more light-emitting diodes.
Clause 6. The end-of-dose indicator device according to clause 4 or clause 5, wherein the housing further comprises one or more windows configured to allow the one or more visual indicators to be seen outside of the housing.
Clause 7. The end-of-dose indicator device according to clause 6, wherein the one or more windows are arranged at the distal end of the housing.
Clause 8. The end-of-dose indicator device according to clause 6, wherein the one or more windows are arranged at the proximal end of the housing.
Clause 9. The end-of-dose indicator device according to clause 6, wherein the one or more windows are arranged circumferentially around the housing.
Clause 10. The end-of-dose indicator device according to clause 9, wherein the one or more windows are arranged circumferentially around the distal end of the housing.
Clause 11. The end-of-dose indicator device according to clause 6, wherein the one or more windows are arranged along a longitudinal axis of the housing between the proximal end of the housing and the distal end of the housing.
Clause 12. The end-of-dose indicator device according to clause 6, wherein the one or more windows are arranged on a proximal-facing surface of the proximal end of the housing.
Clause 13. The end-of-dose indicator device according to any of clauses 1-12, wherein a shape of the housing matches a shape of the proximal end of the medical injection device.
Clause 14. The end-of-dose indicator device according to any of clauses 1-13, wherein the circuit is configured such that activation of the indicator is delayed following closure of the switch.
Clause 15. The end-of-dose indicator device according to clause 14, wherein activation of the indicator is delayed at least 2 seconds.
Clause 16. The end-of-dose indicator device according to clause 14, wherein activation of the indicator is delayed at least 5 seconds.
Clause 17. A medical injection system, comprising: a medical injection device comprising: an injection device housing having a proximal end, a distal end, and a sidewall therebetween defining an injection device interior; a syringe received within the injection device interior, the syringe comprising a proximal end comprising a piston, a distal end comprising a needle, and a sidewall therebetween defining a syringe interior configured to receive a medicament; a plunger rod received within the injection device interior and configured to displace the piston distally to cause the medicament to be expelled through the needle; a drive assembly configured to displace the plunger rod distally; a plunger rod follower comprising a distal end operatively coupled to the plunger rod and a proximal end; and an end-of-dose indicator device, comprising: an indicator housing comprising a proximal end, a distal end, and a sidewall therebetween defining an indicator interior, the indicator housing configured to be secured to the proximal end of the medical injection device; a switch arranged within the indicator housing interior and having an open position and a closed position; a circuit; and one or more indicators in electrical communication with the circuit, wherein the plunger rod follower is configured such when as the plunger rod is displaced distally the proximal end of the plunger rod follower is displaced proximally and contacts the switch, moving the switch from the open position to the closed position and triggering the circuit to activate the indicator.
Clause 18. The system according to clause 17, wherein the indicator housing comprises a cylinder with a flared proximal end.
Clause 19. The system according to clause 18, wherein the flared proximal end comprises a proximal-facing outer surface configured to receive a user's thumb.
Clause 20. The system according to any of clauses 17-19, wherein the one or more indicators are one or more visual indicators.
Clause 21. The system according to clause 20, wherein the one or more visual indicators are one or more light-emitting diodes.
Clause 22. The system according to clause 20, wherein the indicator housing further comprises one or more windows configured to allow the one or more visual indicators to be seen outside of the housing.
Clause 23. The system according to clause 22, wherein the one or more windows are arranged at the distal end of the indicator housing.
Clause 24. The system according to clause 22, wherein the one or more windows are arranged at the proximal end of the indicator housing.
Clause 25. The system according to clause 22, wherein the one or more windows are arranged circumferentially around the indicator housing.
Clause 26. The system according to clause 25, wherein the one or more windows are arranged circumferentially around the distal end of the indicator housing.
Clause 27. The system according to clause 22, wherein the one or more windows are arranged along a longitudinal axis of the indicator housing between the proximal end of the indicator housing and the distal end of the indicator housing.
Clause 28. The system according to clause 22, wherein the one or more windows are arranged on a proximal-facing surface of the proximal end of the indicator housing.
Clause 29. The system according to any of clauses 17-28, wherein a shape of the indicator housing matches a shape of the proximal end of the injection device housing
Clause 30. The system according to any of clauses 17-29, wherein the circuit is configured such activation of the indicator is delayed following closure of the switch.
Clause 31. The system according to clause 30, wherein activation of the indicator is delayed at least 2 seconds.
Clause 32. The system according to clause 30 or clause 31, wherein activation of the indicator is delayed at least 5 seconds.

### BRIEF DESCRIPTION OF THE DRAWINGS

FIG. 1 is a perspective view of an end-of-dose indicator according to non-limiting embodiments or aspects described herein;

FIG. 2 is a front view of an end-of-dose indicator according to non-limiting embodiments or aspects described herein;

FIG. 3 is a side view of an end-of-dose indicator according to non-limiting embodiments or aspects described herein;

FIG. 4 is a top view of an end-of-dose indicator according to non-limiting embodiments or aspects described herein;

FIG. 5 is a bottom view of an end-of-dose indicator according to non-limiting embodiments or aspects described herein;

FIG. 6 is a cross-sectional view along line 6-6 in FIG. 4 of an end-of-dose indicator according to non-limiting embodiments or aspects described herein;

FIG. 7 is a perspective view of an end-of-dose indicator according to non-limiting embodiments or aspects described herein;

FIG. 8 is a perspective view of an end-of-dose indicator according to non-limiting embodiments or aspects described herein;

FIG. 9 is a perspective view of an end-of-dose indicator according to non-limiting embodiments or aspects described herein;

FIG. 10 is a side view of a system including an end-of-dose indicator according to non-limiting embodiments or aspects described herein;

FIG. 11 is a top view of a system including an end-of-dose indicator according to non-limiting embodiments or aspects described herein;

FIG. 12 is a cross-sectional view along line 12-12 in FIG. 11 of a system including an end-of-dose indicator according to non-limiting embodiments or aspects described herein;

FIG. 13 is a cross-sectional view along line 12-12 in FIG. 11 of a system including an end-of-dose indicator according to non-limiting embodiments or aspects described herein; and

FIG. 14 is a cross-sectional view along line 12-12 in FIG. 11 of a system including an end-of-dose indicator according to non-limiting embodiments or aspects described herein.

### DESCRIPTION OF THE INVENTION

The use of numerical values in the various ranges specified in this application, unless expressly indicated otherwise, are stated as approximations as though the minimum and maximum values within the stated ranges are both preceded by the word “about”. As used herein, the term “about” means the stated value ±10%. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

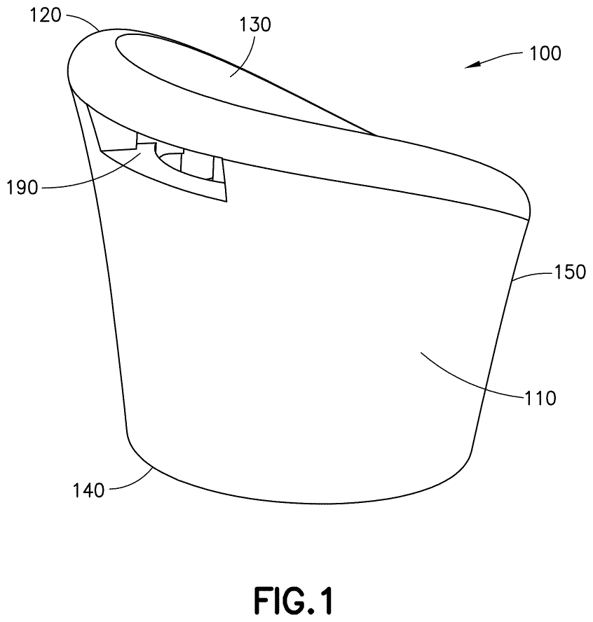

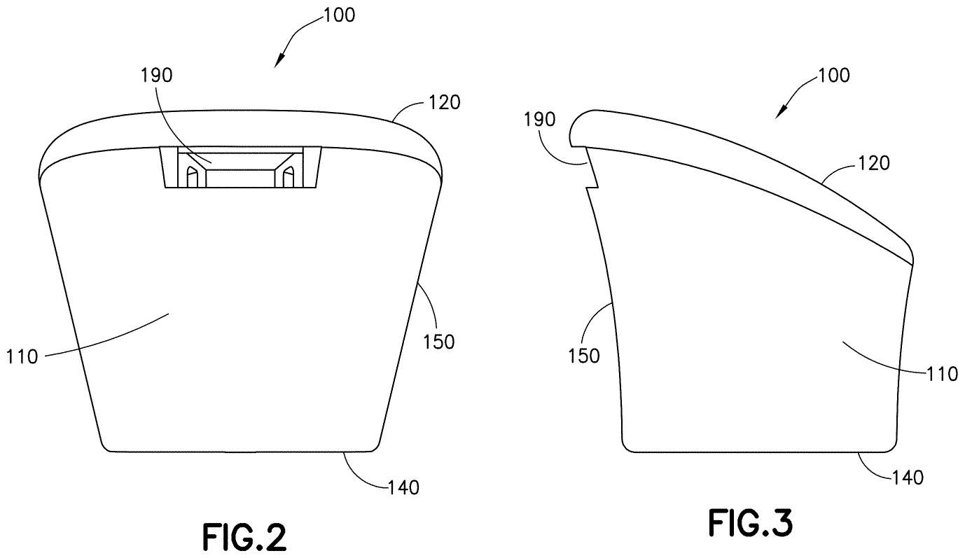

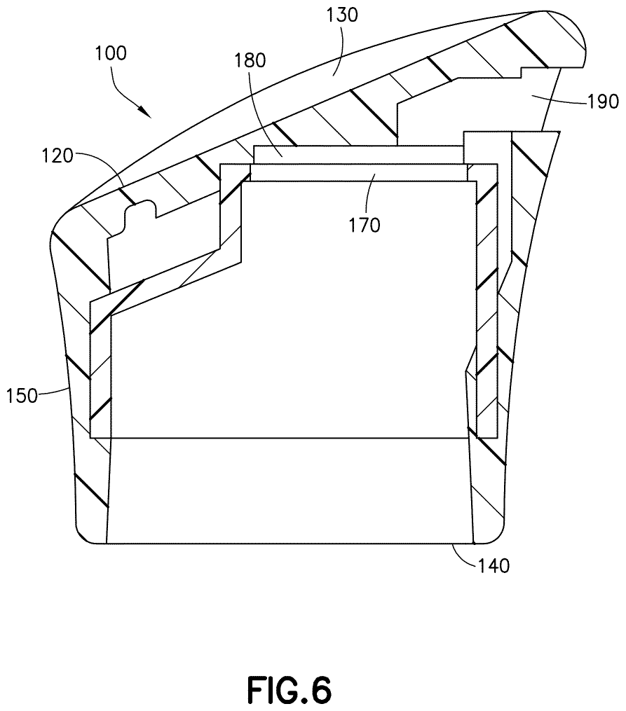

Turning to FIGS. 1-6, provided herein is an end-of dose indicator device 100 for a medical injection device 1000, such as shown in FIG. 11. End-of-dose indicator device 100 includes a housing 110 having a proximal end 120, distal end 140, and sidewall 150 therebetween defining an interior. As an end-of-dose indicator can, in non-limiting embodiments or aspects, be coupled to (reversible or permanently) a medical injection device (not shown), the interior of the housing 110 can, in non-limiting embodiments or aspects, be adapted to receive a portion of the medical injection device therein.

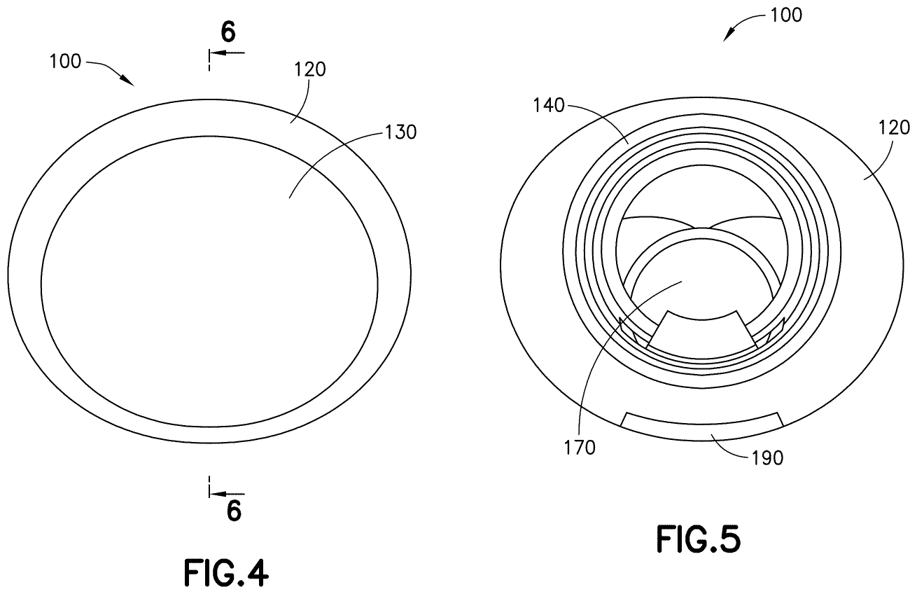

Housing 110 can assume any useful configuration to allow coupling to a medical injection device (not shown). In non-limiting embodiments or aspects, housing 110, when viewed in cross-section from the proximal end 120 or distal end 140 thereof (e.g., FIG. 4), is substantially cylindrical, cylindrical, has an oval shape, and/or has the shape of an ellipse. In non-limiting embodiments or aspects, proximal end 120 is flared outward of the circumference of the sidewall 150. In non-limiting embodiments or aspects, proximal end 120 has a proximal-facing outer surface 130. Proximal-facing outer surface 130 can assume any suitable configuration, for example, to provide an ergonomic characteristic to the medical injection device and/or increase stability during the injection process. In non-limiting embodiments or aspects, proximal-facing outer surface 130 can be angled to allow for a thumb of a user of a medical injection device (not shown) to which end-of-dose injection device 100 is coupled to be placed thereon, for example, to provide a surface with which the user can apply force when applying the medical injection device a site of injection.

Housing 110 can be formed of any suitable material. In non-limiting embodiments, housing 110 is formed of polymeric materials, natural or synthetic rubber materials, metals, alloys, and/or combinations thereof. In non-limiting embodiments or aspects, proximal-facing outer surface 130 of housing 110 can include one or more resilient and/or elastomeric materials to provide an ergonomic feel, such as elastomeric polymers, synthetic and/or natural rubbers, foams, and combinations thereof.

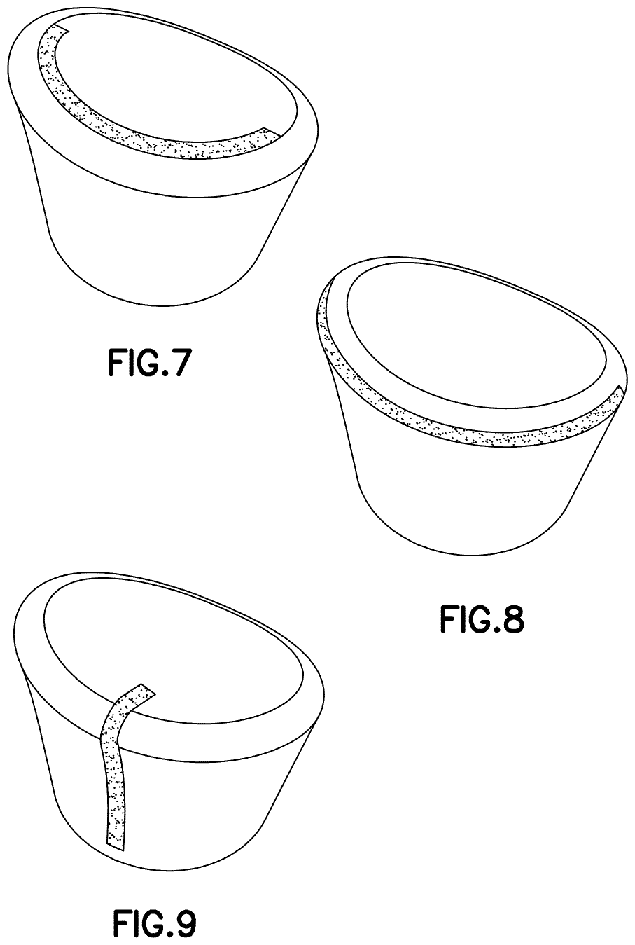

With continuing reference to FIGS. 1-9, end-of-dose indicator device 100 includes a switch 160, a circuit 170, and an indicator 180 arranged within the interior of housing 110. Switch 160 and circuit 170 can be arranged as is known in the art, such that switch 160 can assume an open configuration, in which current cannot pass through the circuit, and a closed configuration, in which current can pass through the circuit. End-of-dose indicator device 100 can include an electrochemical cell, such as a battery (not shown), in electrical communication with circuit 170. Circuit 170 can include one or more batteries, wires, resistors, transistors, capacitors, fuses, speakers, haptic devices, and/or diodes. In non-limiting embodiments or aspects, indicator 180 is in electrical communication with and/or is part of circuit 170. In non-limiting embodiments or aspects, indicator 180 is an audible indicator, a tactile indicator, and/or a visual indicator. In non-limiting embodiments or aspects, indicator 180 is a diode, such as a photodiode. In non-limiting embodiments or aspects, indicator 180 is a light-emitting diode (LED).

In non-limiting embodiments, circuit 170 is a delay circuit, in that circuit 170 is configured, for example, with one or more capacitors, such that there is a delay between closure of switch 160 and supply of power to indicator 180. Such a delay provides an interval, for example, a pre-determined interval, between the end of delivery of medicament and the initiation of the indication. Those of skill in the art will appreciate that any suitable delay can be included based on basic circuity characteristics. In non-limiting aspects or embodiments, circuit 170 is configured to provide a delay between switch closure and initiation of the indication by at about 1, about 2, about 3, about 4, or about 5 seconds, all values and subranges therebetween inclusive.

In non-limiting embodiments or aspects, housing 110 can include one or more windows 190. One or more windows 190 can be arranged on housing 110 in any desired configuration. One or more windows 190 can be one or more openings in housing 110, and/or can include a translucent and/or transparent material that allows light to at least partially pass therethrough. In non-limiting embodiments or aspects, one or more windows 190 are arranged at proximal end 120, distal end 140, on proximal-facing outer surface 130 of housing 110, longitudinally between proximal end 120 and distal end 140 of housing 110, circumferentially about housing 110, and/or any combination thereof. In non-limiting embodiments or aspects, windows 190 are arranged as shown in FIGS. 1-3 and/or 7-9.

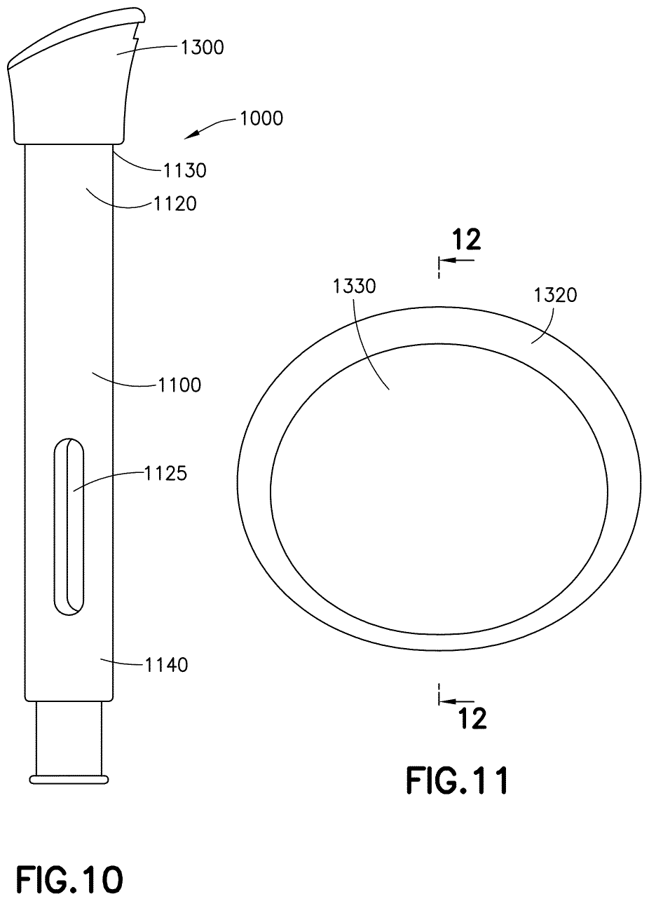

With reference to FIGS. 10-14, shown is a system 1000 including a medical injection device 1100 and an end-of-dose indicator device 1300 as described herein. While medical injection device 1100 is shown with minimal internal components, those of skill in the art will appreciate that any type of medical injection device, for example, those where the syringe 1170 is actively inserted at the injection site by a drive assembly 1250, those where the syringe 1170 is retracted following delivery of the medicament 1230, those where a button is used to deliver medicament 1230, and those where no button is required (e.g., “push-on” activation) can be used with the end-of-dose indicator device described herein.

Medical injection device 1100 can include an injection device housing 1120 having a proximal end 1130, a distal end 1140, and a sidewall 1160 therebetween defining an interior. Injection device housing 1120 can be a two-piece housing, for example, a proximal part and a distal part that can be assembled. Injection device housing 1120 can also include a window 1125.

Medical injection device 1100 can further include a syringe 1170 received in the interior thereof, and syringe 1170 can include a proximal end 1180, a distal end 1200, and a sidewall 1220 therebetween defining an interior configured to contain a composition, such as a medicament 1230. Syringe 1170 can further include at distal end 1200 thereof a needle 1210 with one or more openings therein for delivery of medicament 1230. Needle 1210 can be formed of any suitable material, and can be of any useful gauge. Syringe 1170 can further include at proximal end 1180 thereof a piston 1190. Piston 1190 can be configured to seal the interior of syringe 1170, and can be displaced distally to force medicament 1230 through needle 1210 to a site of injection. Piston 1190 can be formed of a natural rubber, a synthetic rubber, or mixtures thereof. In some aspects, the second component is formed of one or more of butyl rubber (including copolymers of isobutylene (about 97-98%) and isoprene (about 2-3%)), chlorinated butyl rubber, brominated butyl rubber, bromides of an isobutylene-paramethylstyrene copolymer, polyisoprene rubber, polybutadiene rubber, styrene ethylene butylene (SBS) rubber, styrene-isoprene (SIS) block polymers or styrene-isoprene/butadiene (SIBS), in which the content of styrene in the styrene block copolymer ranges from about 10% to about 70%, and preferably from about 20% to about 50%, epichlorohydrin rubber, styrene-butadiene (SBR) rubber, and mixtures thereof.

Syringe 1170 can be a pre-filled syringe, as is known in the art, and can include coatings, such as those including polydimethylsiloxane (PDMS) and/or other polymers, on sidewall 1220 and/or piston 1190 to reduce friction and/or reduce interactions between syringe 1170 components and compositions held within syringe 1170. In non-limiting embodiments, syringe 1170 is a unitary syringe, and needle 1210 is formed with the syringe barrel. In non-limiting embodiments or aspects, syringe 1170 includes a needle hub, which holds needle 1210. Syringe 1170 can be formed of any useful material, for example, plastic or glass, and piston 1190 can be formed of suitable materials such as natural and/or synthetic rubbers, polymeric materials, and the like.

In non-limiting embodiments or aspects, syringe 1170 can include a needle shield (not shown). In non-limiting embodiments or aspects, needle shield can be a one-piece needle shield formed of an elastomeric material, or a two-piece needle shield having an inner portion formed of an elastomeric material and an outer portion formed of a rigid material, such as a rigid polymeric material. Such needle shields are commonly referred to as rigid needle shields (RNS).

With continuing reference to FIGS. 10-14, medical injection device 1100 can include plunger rod 1240 having a proximal end 1244 and a distal end 1246. Plunger rod distal end 1246 can be configured to interact with piston 1190 of syringe 1170, to displace piston 1190 distally to deliver medicament through needle 1210. Medical injection device 1100 can also include a drive assembly 1250 configured to bias plunger rod 1240 distally. While drive assembly 1250 is exemplified in FIGS. 10-14 as a spring coupled to plunger rod 1240, those of skill in the art will appreciate that any suitable mechanism for displacing plunger rod 1240 distally can be used.

Medical injection device 1100 can further include a plunger rod follower 1260, having a proximal end, a distal end, and a protrusion 1270 extending proximally from proximal end. Plunger rod follower 1260 can be coupled to or otherwise interact with proximal end 1244 of plunger rod 1240. In non-limiting embodiments, plunger rod follower 1260 is configured to engage with plunger rod 1240 and injection device housing 1120 to cause plunger rod follower 1260 to move proximally once plunger rod 1240 has moved distally a certain amount. For example, and without limitation, proximal end 1244 of plunger rod 1240 can be received within an opening in plunger rod follower 1260, applying a radial force and engaging plunger rod follower 1260 with an inner wall of injection device housing 1120 through a friction fit between plunger rod follower 1260 and injection device housing 1120 or through interaction of plunger rod follower 1260 and one or more protrusions 1270 extending inward from housing 1120. This arrangement maintains proximal end of plunger rod follower spaced from proximal end 1130 of injection device housing and maintains protrusion 1270 within the interior of injection device housing 1120 and/or within opening 1150 in proximal end 1230 of injection device housing 1120.

As plunger rod 1240 moves distally during delivery of a medicament 1230, proximal end 1244 thereof is pulled out of engagement with plunger rod follower 1260, allowing plunger rod follower 1260, in non-limiting embodiments or aspects, to move radially inward and out of interaction with injection device housing 1120. Arrangement of the interior of injection device housing 1120 at proximal end 1130 thereof can cause plunger rod follower 1260 to move proximally, and cause protrusion 1270 to pass through opening 1150 in proximal end 1230 of injection device housing 1120. In non-limiting embodiments or aspects, movement of plunger rod follower 1260 proximally can cause plunger rod follower 1260 to contact proximal end 1130 of injection device housing 1100, causing an audible and/or tactile indication. In non-limiting embodiments or aspects, no such indication is provided, so as to reduce confusion and remove a false indication that may cause the user to remove the injection device 1100 from the site of injection before the injection is completed.

System 1000 also can include an end-of dose indicator device 1300 as described above. End-of-dose indicator device 1300 includes a housing 1310 having a proximal end 1320, distal end 1340, and sidewall 1350 therebetween defining an interior. As shown in FIGS. 10-14, end-of-dose indicator 1300 is coupled to (reversible or permanently) medical injection device 1100, and the interior of the housing 1310 is adapted to receive a portion of the medical injection device 1100 therein. For example, and without limitation, end-of-dose indicator device 1300 can be a two-piece component that snaps together and surrounds proximal end 1130 of injection device housing 1120, end-of-dose indicator device 1300 can be held on proximal end 1130 of injection device housing 1120 through a friction fit, a snap fit to a portion of indicator housing 1120, or like known attachment mechanism. In non-limiting embodiments or aspects, indicator device is co-molded with injector device housing 1120, and/or has a unitary construction with housing 1120.

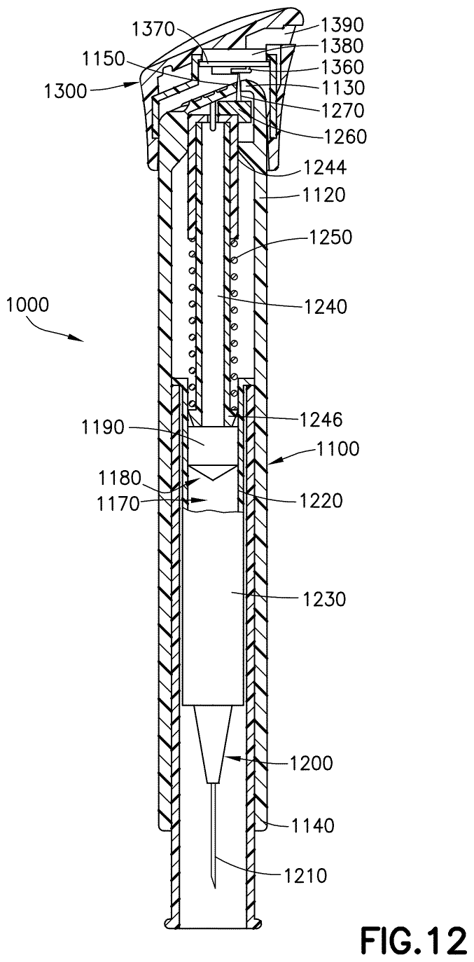

With continuing reference to FIGS. 12-14, end-of-dose indicator device 1300 includes a switch 1360, a circuit 1370, and/or an indicator 1380, each as described above, arranged within the interior of housing 1310. In non-limiting embodiments or aspects, indicator 1380 is an audible indicator, a tactile indicator, and/or a visual indicator. In non-limiting embodiments or aspects, indicator 1380 is a diode, such as a photodiode. In non-limiting embodiments or aspects, indicator 1380 is an LED.

In non-limiting embodiments, circuit 1370 is a delay circuit as described above, in that circuit 1370 is configured, for example, with one or more capacitors, such that there is a delay between closure of switch 1360 and supply of power to indicator 1380. In non-limiting aspects or embodiments as described above, circuit 1370 is configured to provide a delay between switch closure and initiation of the indication by at about 1, about 2, about 3, about 4, or about 5 seconds, all values and subranges therebetween inclusive.

In non-limiting embodiments or aspects, housing 1310 can include one or more windows 1390. One or more windows 1390 can be arranged on housing 1310 in any desired configuration. One or more windows 1390 can be one or more openings in housing 1310, and/or can include a translucent and/or transparent material that allows light to at least partially pass therethrough. In non-limiting embodiments or aspects, one or more windows 1390 are arranged at proximal end 1320, distal end 1340, on proximal-facing surface 1330 of housing 1310, longitudinally between proximal end 1320 and distal end 1340 of housing 1310, circumferentially about housing 1310, and/or any combination thereof, for example, as described above and as shown in FIGS. 1-3 and/or 7-9.

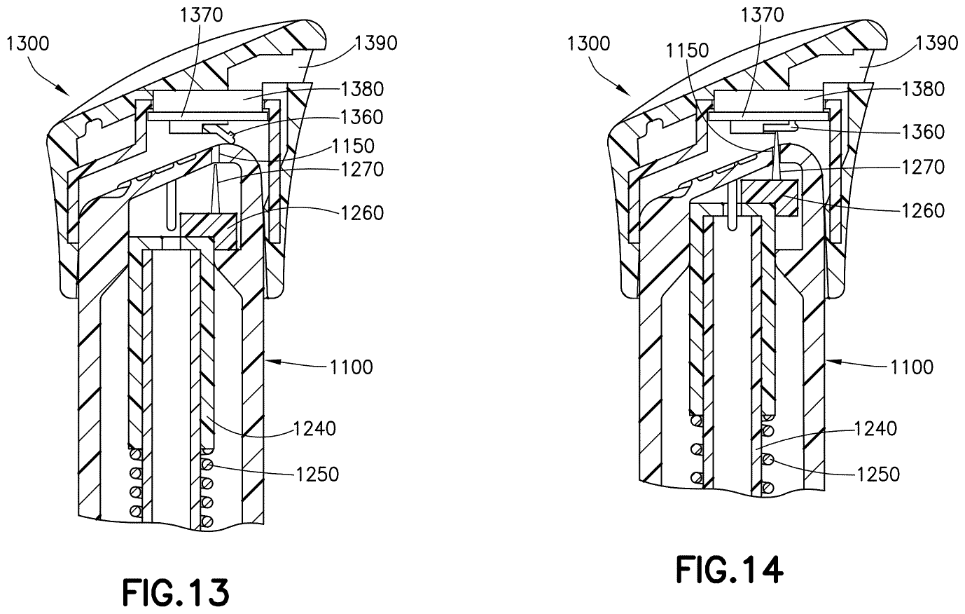

With reference to FIGS. 13 and 14, shown is an enlarged view of the proximal end of system 1000 before/during plunger rod 1240 displacement and following displacement. As can be appreciated, through movement of plunger rod follower 1260 protrusion 1270 proximally, protrusion 1270 can pass through opening 1150 in proximal end 1230 of injection device housing 1120. In doing so, protrusion 1270 can contact switch 1360 and move switch 1360 to a closed position, allowing current to accumulate/flow and for indicator 1380 to be activated.

In non-limiting embodiments or aspects, medical injection device 1100 can also include a removable cap (not shown). In non-limiting embodiments or aspects, removable cap can be configured to remove any needle shield, such as an RNS, that is included with syringe 1170. In non-limiting embodiments or aspects, the cap is a two-piece cap, one portion of the two-piece cap (e.g., a cap remover) configured to be grasped by a used and pulled axially away from distal end 1140 of injection device housing and a second portion (e.g., a retainer) configured to grasp and remove a needle shield, such as an RNS (not shown).

Although the devices and systems have been described in detail for the purpose of illustration based on what is currently considered to be the most practical and preferred embodiments, it is to be understood that such detail is solely for that purpose and that the systems and methods are not limited to the disclosed embodiments, but on the contrary, is intended to cover modifications and equivalent arrangements that are within the spirit and scope of the appended claims. For example, it is to be understood that the present systems and methods contemplate that, to the extent possible, one or more features of any embodiment can be combined with one or more features of any other embodiment.

1. An end-of-dose indicator device for a medical injection device, comprising:
a housing comprising a proximal end, a distal end, and a sidewall therebetween defining an interior, the housing configured to be secured to a proximal end of a medical injection device;
a switch arranged within the housing interior and having an open position and a closed position;
a circuit; and
one or more indicators in electrical communication with the circuit,
wherein the switch, when in the closed position, triggers the circuit to activate the indicator.

2. The end-of-dose indicator device according to claim 1, wherein the housing comprises a cylinder with a flared proximal end.

3. The end-of-dose indicator device according to claim 2, wherein the flared proximal end comprises a proximal-facing outer surface configured to receive a user's thumb.

4. The end-of-dose indicator device according to claim 1, wherein the one or more indicators are one or more visual indicators.

5. The end-of-dose indicator device according to claim 4, wherein the one or more visual indicators are one or more light-emitting diodes.

6. The end-of-dose indicator device according to claim 4, wherein the housing further comprises one or more windows configured to allow the one or more visual indicators to be seen outside of the housing.

7. The end-of-dose indicator device according to claim 6, wherein the one or more windows are arranged at the distal end of the housing.

8. The end-of-dose indicator device according to claim 6, wherein the one or more windows are arranged at the proximal end of the housing.

9. The end-of-dose indicator device according to claim 6, wherein the one or more windows are arranged circumferentially around the housing.

10. The end-of-dose indicator device according to claim 9, wherein the one or more windows are arranged circumferentially around the distal end of the housing.

11. The end-of-dose indicator device according to claim 6, wherein the one or more windows are arranged along a longitudinal axis of the housing between the proximal end of the housing and the distal end of the housing.

12. The end-of-dose indicator device according to claim 6, wherein the one or more windows are arranged on a proximal-facing surface of the proximal end of the housing.

13. The end-of-dose indicator device according to claim 1, wherein a shape of the housing matches a shape of the proximal end of the medical injection device.

14. The end-of-dose indicator device according to claim 1, wherein the circuit is configured such that activation of the indicator is delayed following closure of the switch.

15. The end-of-dose indicator device according to claim 14, wherein activation of the indicator is delayed at least 2 seconds.

16. The end-of-dose indicator device according to claim 14, wherein activation of the indicator is delayed at least 5 seconds.

17. A medical injection system, comprising:
a medical injection device comprising:
an injection device housing having a proximal end, a distal end, and a sidewall therebetween defining an injection device interior;
a syringe received within the injection device interior, the syringe comprising a proximal end comprising a piston, a distal end comprising a needle, and a sidewall therebetween defining a syringe interior configured to receive a medicament;
a plunger rod received within the injection device interior and configured to displace the piston distally to cause the medicament to be expelled through the needle;
a drive assembly configured to displace the plunger rod distally;
a plunger rod follower comprising a distal end operatively coupled to the plunger rod and a proximal end; and
an end-of-dose indicator device, comprising:
an indicator housing comprising a proximal end, a distal end, and a sidewall therebetween defining an indicator interior, the indicator housing configured to be secured to the proximal end of the medical injection device;
a switch arranged within the indicator housing interior and having an open position and a closed position;
a circuit; and
one or more indicators in electrical communication with the circuit,
wherein the plunger rod follower is configured such when as the plunger rod is displaced distally the proximal end of the plunger rod follower is displaced proximally and contacts the switch, moving the switch from the open position to the closed position and triggering the circuit to activate the indicator.

18. The system according to claim 17, wherein the indicator housing comprises a cylinder with a flared proximal end.

19. The system according to claim 18, wherein the flared proximal end comprises a proximal-facing outer surface configured to receive a user's thumb.

20. The system according to claim 17, wherein the one or more indicators are one or more visual indicators.

21. The system according to claim 20, wherein the one or more visual indicators are one or more light-emitting diodes.

22. The system according to claim 20, wherein the indicator housing further comprises one or more windows configured to allow the one or more visual indicators to be seen outside of the housing.

23. The system according to claim 22, wherein the one or more windows are arranged at the distal end of the indicator housing.

24. The system according to claim 22, wherein the one or more windows are arranged at the proximal end of the indicator housing.

25. The system according to claim 22, wherein the one or more windows are arranged circumferentially around the indicator housing.

26. The system according to claim 25, wherein the one or more windows are arranged circumferentially around the distal end of the indicator housing.

27. The system according to claim 22, wherein the one or more windows are arranged along a longitudinal axis of the indicator housing between the proximal end of the indicator housing and the distal end of the indicator housing.

28. (canceled)

29. (canceled)

30. The system according to claim 17, wherein the circuit is configured such activation of the indicator is delayed following closure of the switch.

31. The system according to claim 17, wherein activation of the indicator is delayed at least 2 seconds.

32. The system according to claim 3, wherein activation of the indicator is delayed at least 5 seconds.
